(19) United States
(12) Patent Application Publication
(10) Pub. No.: US 2009/0264770 A1
(43) Pub. Date: Oct. 22, 2009

(54) Medical Systems and Related Methods

(75) Inventors: Clifford M. Liu, Randolph, MA (US); James Edward Barrington, Lexington, MA (US)

Correspondence Address: FISH & RICHARDSON PC, P.O. BOX 1022, MINNEAPOLIS, MN, 55440-1022, US

(73) Assignee: OMNISONICS MEDICAL TECHNOLOGIES, INC., Wilmington, MA (US)

(21) Appl. No.: 12/104,635

(22) Filed: Apr. 17, 2008

(51) Int. Cl. A61B 8/14

(52) U.S. Cl. 600/466

(57) ABSTRACT

This disclosure relates to medical systems and related methods. In some embodiments, the medical systems include a catheter having a coiled section that is radially displaceable relative to a waveguide.

### TECHNICAL FIELD

This disclosure relates to medical systems and related methods.

### BACKGROUND

An ultrasound medical system can be used to treat a subject (e.g., a human) having certain conditions. Typically, a distal portion of the ultrasound medical system is disposed within the subject and then the system is activated so that the distal portion of the system vibrates at an ultrasonic frequency. The ultrasonic vibrations emitted by the distal portion of the system can treat certain conditions by breaking up unwanted tissue in the subject.

### SUMMARY

In one aspect of the invention, a medical system includes a catheter having a coiled section that defines a first lumen and a second lumen that extends axially through the coiled section. In addition, the medical system includes a waveguide disposed within the second lumen. The coiled section of the catheter is radially displaceable relative to the waveguide.

In another aspect of the invention, a medical system includes a catheter that defines a lumen and a guidewire configured to be disposed within the lumen of the catheter. At least a portion of the guidewire is configured to have a non-coiled configuration at a first temperature and a coiled configuration at a second temperature such that when the guidewire is at the second temperature and disposed within the lumen of the catheter, a coiled shape is imparted to the catheter.

In an additional aspect of the invention, a method includes expanding a section of a catheter within a body vessel and vibrating a waveguide disposed within a lumen defined by the expanded section of the catheter. The expanded section of the catheter is in the form of a coil.

Embodiments can include one or more of the following features.

In some embodiments, the first lumen is a helical-shaped lumen.

In certain embodiments, the coiled section of the catheter includes a helical-shaped tubular member that defines the helical-shaped lumen.

In some embodiments, the catheter further includes a proximal section disposed proximal to the coiled section and a distal section disposed distal to the coiled section, and the coiled section can be radially displaced by axially displacing at least one of the proximal and distal sections relative to the waveguide.

In certain embodiments, the proximal section of the catheter defines a lumen that is substantially aligned with the first lumen of the coiled section.

In some embodiments, the distal section of the catheter defines a lumen that is substantially aligned with the first lumen of the coiled section.

In certain embodiments, the proximal and distal sections of the catheter are substantially linear.

In some embodiments, the coiled section of the catheter is configured such that the coiled section assumes a first level of radial expansion when a guidewire is disposed within the first lumen and a second level of radial expansion when the guidewire is removed from the first lumen. The first level of radial expansion is less than the second level of radial expansion.

In certain embodiments, the coiled section is substantially radially unexpanded when the coiled section is at the first level of radial expansion.

In some embodiments, the medical system further includes a guidewire disposed within the first lumen. The coiled section can be radially displaced relative to the waveguide by axially displacing the guidewire relative to the coiled section.

In certain embodiments, the guidewire is axially displaceable between a first position in which the guidewire is disposed in the first lumen and retains the coiled section in a radially compressed condition, and a second position in which the guidewire is removed from the first lumen and the coiled section is allowed to radially expand.

In some embodiments, the coiled section can be radially expanded relative to the waveguide by proximally displacing the guidewire relative to the coiled section.

In certain embodiments, the coiled section is resiliently biased toward a radially expanded position.

In some embodiments, the coiled section is radially displaceable relative to the waveguide between the radially expanded position and a radially compressed position.

In certain embodiments, the coiled section includes one or more thermoset polymers.

In some embodiments, the medical system further includes an outer sheath at least partially surrounding the coiled section of the catheter. The coiled section of the catheter can be radially displaced relative to the waveguide by axially displacing the outer sheath relative to the coiled section of the catheter.

In certain embodiments, the outer sheath is axially displaceable between a first position in which the outer sheath at least partially surrounds the coiled section and retains the coiled section in a radially compressed condition, and a second position in which the coiled section is allowed to radially expand.

In some embodiments, the coiled section of the catheter can be radially expanded relative to the waveguide by proximally displacing the outer sheath relative to the coiled section.

In certain embodiments, the coiled section of the catheter includes multiple helical-shaped members.

In some embodiments, the medical system further includes a guidewire configured to fit within the first lumen. At least a portion of the guidewire is adapted to be formed into the shape of a coil upon heating the guidewire above a predetermined temperature.

In certain embodiments, the guidewire includes one or more shape-memory materials.

In some embodiments, a distal end portion of the waveguide is fixedly or movably secured to the catheter.

In certain embodiments, the coiled section of the catheter can be radially displaced relative to the waveguide by axially displacing the distal end portion of the waveguide.

In some embodiments, the coiled section of the catheter includes a helical-shaped tubular member that defines a helical opening along the coiled section of the catheter.

In certain embodiments, the helical-shaped tubular member is configured such that the helical opening has a first width in a first portion of the coiled section and a second width in a second portion of the coiled section, the first width being greater than the second width.

In some embodiments, the first portion of the coiled section is a proximal end region of the coiled section and the second portion of the coiled section is a distal end region of the coiled section.

In certain embodiments, the medical system further includes a polymeric film surrounding an end portion of the coiled section.

In some embodiments, the medical system further includes a waveguide at least partially surrounded by the catheter.

In certain embodiments, the second temperature is higher than the first temperature.

In some embodiments, the expanded section of the catheter is disposed within an at least partially occluded region of the body vessel.

In certain embodiments, the expanded section of the catheter includes one or more helical-shaped tubular members.

In some embodiments, expanding the section of the catheter includes proximally displacing an outer sheath relative to the section of the catheter.

In certain embodiments, expanding the section of the catheter includes axially displacing the outer sheath from a first position in which the outer sheath at least partially surrounds the section of the catheter and retains the section of the catheter in a radially compressed condition, toward a second position in which the outer sheath is axially spaced apart from the section of the catheter.

In some embodiments, expanding the section of the catheter includes proximally displacing a guidewire relative to the section of the catheter.

In certain embodiments, proximally displacing the guidewire includes axially displacing the guidewire from a first position in which the guidewire is disposed within a guidewire lumen defined by the section of the catheter, toward a second position in which the guidewire is removed from the guidewire lumen defined by the section of the catheter.

In some embodiments, expanding the section of the catheter includes heating a guidewire disposed within a guidewire lumen defined by the section of the catheter.

In certain embodiments, heating the guidewire includes passing a heated fluid through the catheter.

In some embodiments, heating the guidewire includes applying electrical energy to the guidewire.

In certain embodiments, a portion of the waveguide disposed within the expanded section of the catheter is transversely vibrated.

Embodiments can include one or more of the following advantages.

In some embodiments, the system is configured to inhibit direct physical contact between the vibrating waveguide and the body vessel during treatment. As a consequence, the dampening effect on the waveguide resulting from direct contact between the waveguide and the surrounding environment can be reduced, which can lead to increased efficiency and increased waveguide life cycles. The ability of the system to inhibit the waveguide from contacting the wall of the body vessel can be particularly advantageous when the system is operating within a tortuous region of the body vessel since the waveguide tends to bow radially outward in such cases.

In certain embodiments, the system is configured so that, in the coiled section of the catheter, the waveguide only contacts the catheter at longitudinally spaced points. For example, when the coiled section of the catheter is disposed within a tortuous region of a body vessel such that the waveguide bows radially outward, contact between the waveguide and the coiled section of the catheter is limited to longitudinally spaced points along the coiled section of the catheter. By limiting the area of the catheter that is contacted by the waveguide during use, stress on the waveguide can be reduced, resulting in increased waveguide life cycles.

In some embodiments, the system is configured to limit axial movement of the waveguide. For example, the coiled section of the waveguide can provide a radial boundary for the waveguide during use. By limiting the extent to which the waveguide can move radially (e.g., as a result of bowing radially outward within a tortuous region of a body vessel), axial movement of the distal end region of the waveguide in the proximal direction is also limited. This arrangement can help to ensure that a distal end portion of the waveguide stays within a lumen of a distal section of the catheter during use and does not move proximally into the axial lumen defined by the coiled section of the catheter. As a result, the distal end portion of the waveguide can be inhibited from popping out of an opening defined by the coiled section (e.g., defined by a distal region of the coiled section) of the catheter and contacting the wall of the body vessel as the catheter and waveguide are navigated through the body vessel and/or as the system is operated within the body vessel.

In certain embodiments, the system provides for three dimensional transmission of vibrational energy. For example, the system can permit the simultaneous transmission of vibrational energy in multiple radial directions (e.g., in all radial directions about the 360 degree circumference of the waveguide) via a helical opening extending along the coiled section of the catheter. This can result in reduced operating procedure time, and, as a result, reduced energy input and increased waveguide life cycle.

In some embodiments the system can aid in centering the waveguide within the body lumen. As a result, vibrational energy can be delivered with approximately equal intensity to body tissue surrounding the waveguide at multiple circumferential points (e.g., at all circumferential points) around the waveguide. This can, for example, inhibit spotty removal of body tissue and help to ensure that body tissue is removed around the entire circumference of the waveguide during treatment.

In certain embodiments, the system is configured so that a section of the catheter can be radially displaced by inserting a guidewire into a lumen in the catheter and/or by removing a guidewire from a lumen in the catheter. The guidewire can, for example, be heat-activated so that, upon heating the guidewire, a deformable section of the guidewire is formed into the shape of a coil, and thus imparts a coiled shape to the section of the catheter in which it is disposed. Alternatively or additionally, a section of the catheter can be biased toward a coiled shape. In such cases, the coil-shaped section of the catheter can be radially contracted by disposing a relatively rigid guidewire within a lumen of the coiled section, and the coiled section can be allowed to radially expand by retracting the relatively rigid guidewire from the lumen of the coiled section. Thus, a section of the catheter can be radially displaced with only minor modifications to certain traditional treatment methods.

Other aspects, features, and advantages are in the description, drawings, and claims.

### DESCRIPTION OF DRAWINGS

FIG. 1 is a schematic, broken side view of a medical system including a catheter having a coiled section surrounding a waveguide.

FIG. 2 is a side view of the waveguide of the medical system of FIG. 1.

FIGS. 3A-3C are cross-sectional views of the medical system of FIG. 1, taken along lines 3A-3A, 3B-3B, and 3C-3C, respectively, in FIG. 1.

FIGS. 4A-4C illustrate a method of controlling radial displacement of the coiled section of the catheter of the medical system of FIG. 1.

FIGS. 5A and 5B illustrate a method of making the catheter of the medical system of FIG. 1.

FIGS. 6A-6E illustrate a method of using the medical system of FIG. 1 to treat a partially occluded region of a blood vessel.

FIG. 7 illustrates the medical system of FIG. 1 being used to treat a partially occluded, tortuous region of a blood vessel.

FIGS. 8A and 8B are side views of a distal portion of a medical system including a catheter having a coiled section made up of multiple helical-shaped members. The coiled section is shown in a radially expanded state in FIG. 8A and in a radially contracted state in FIG. 8B.

FIGS. 9A and 9B are side views of a medical system including a catheter having a coiled section and a retractable outer sheath surrounding a portion of the catheter. The catheter is shown disposed over the coiled section of the catheter in FIG. 9A such that the coiled section is in a radially compressed state, and the catheter is shown retracted from the coiled section of the catheter in FIG. 9B such that the coiled section is in a radially expanded state.

FIG. 10 is a side view of a distal portion of a medical system including a catheter that at least partially surrounds a waveguide and a shape memory guidewire.

FIGS. 11A and 11B show side views of the shape memory guidewire of the medical system of FIG. 10 in a straight configuration and in a coiled configuration, respectively.

FIGS. 12A and 12B show side views of the distal portion of the medical system of FIG. 10 in a radially contracted configuration and in a radially expanded configuration, respectively.

FIG. 13 is a side view of a distal portion of a medical system including an expanded coiled section adapted for use as a filter.

FIG. 14 is a side view of the distal portion of the medical system of FIG. 13 with a polymeric film surrounding a distal end portion of the expanded coiled section.

FIG. 15 is a side view of a catheter having proximal and distal sections that are axially offset from one another.

FIG. 16 illustrates a device for forming the catheter of FIG. 15.

### DETAILED DESCRIPTION

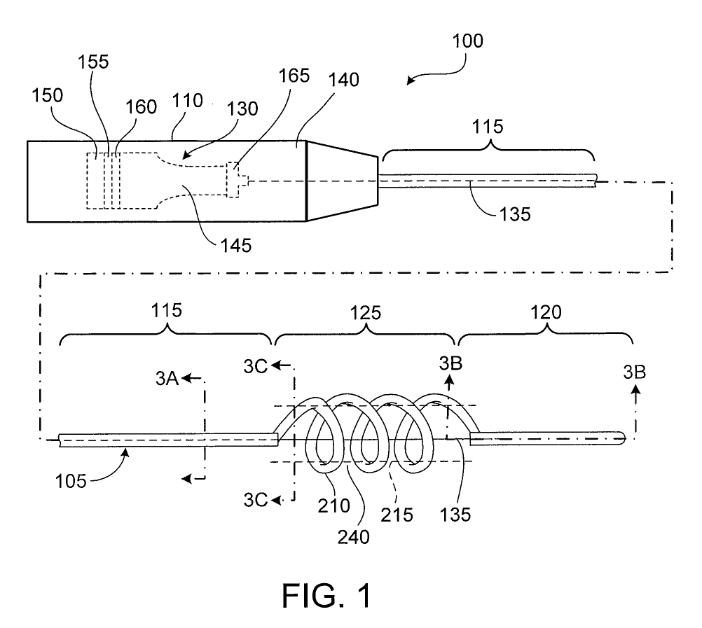

Referring to FIG. 1, a medical system 100 includes a catheter 105 that is secured to a handpiece 110. The catheter 105 includes a proximal section 115, a distal section 120, and a coiled section 125 disposed between the proximal and distal sections 115, 120. The coiled section 125 of the catheter 105 surrounds a waveguide 135 that extends within a lumen of the catheter 105. The handpiece 110 includes an acoustic assembly 130 that is coupled to a proximal end portion of the waveguide 135. During use, electrical energy is delivered from an electrical energy source to the acoustic assembly 130, causing the acoustic assembly 130 to vibrate. As a result, the portion of the waveguide 135 disposed within the coiled section 125 of the catheter 105 vibrates transversely and radially emits vibrational energy. The coiled section 125 of the catheter 105 inhibits the vibrating waveguide 135 from extending radially beyond the coiled section 125 while permitting the vibrational energy emitted by the portion of the waveguide 135 disposed within the coiled section 125 to pass radially through a helical opening 240 extending along the coiled section 125. The coiled section 125 of the catheter 105 can thus inhibit contact between the waveguide 135 and a blood vessel wall during treatment within the blood vessel.

As illustrated in FIG. 1, the handpiece 110 includes a housing 140 in which the acoustic assembly 130 is disposed. The acoustic assembly 130 includes a horn 145, a back mass 150, and piezoceramic rings 155, 160 compressed between the horn 145 and the back mass 150. A bolt can, for example, be used to compress the transducer rings 155, 160 between the horn 145 and the back mass 150. A coupler 165 is secured to the proximal end region of the waveguide 135 and couples the waveguide 135 to the acoustic assembly 130. A distal portion of the coupler 165 includes a bore in which the proximal end region of the waveguide 135 is received. The distal portion of the coupler 165 is crimped around the proximal end region of the waveguide 135 to secure the waveguide 135 to the coupler 165. A proximal portion of the coupler 165 is releasably secured to the horn 145 of the acoustic assembly 130 to releasably secure the waveguide 135 to the horn 145. The proximal portion of the coupler 165 can, for example, include threads that mate with threads on the horn 145 to releasably secure the coupler 165 and waveguide 135 to the horn 145. During use, the transducer rings 155, 160 are electrically connected to an electrical power supply via electrical leads. Upon supplying electrical energy to the transducer rings 155, 160, the transducer rings vibrate, causing the horn 145, the coupler 165, and the waveguide 135 to vibrate longitudinally.

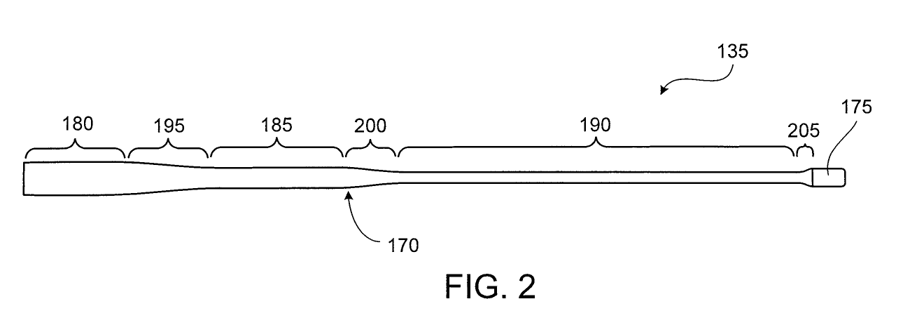

The waveguide 135 is configured to convert the longitudinal vibrational energy transmitted to the waveguide 135 by the acoustic assembly 130 into transverse vibrational energy that is emitted from the portion of the waveguide 135 disposed within the coiled section 125 of the catheter 100. FIG. 2 shows a more detailed view of the waveguide 135. As shown in FIG. 2, the waveguide 135 includes an elongate body member 170 and a radiopaque tip 175 welded to the distal end of the body member 170. The body member 170 is a unitary member formed of Ti-6Al-4V titanium alloy, and the radiopaque tip 175 is formed of tantalum. The body member 170 includes first, second, and third regions 180, 185, 190 of substantially uniform cross-sectional dimension along their lengths. Tapered regions 195, 200 taper distally to smaller diameters and extend between the first and second regions 180, 185 and the second and third regions 185, 190, respectively. Another tapered region 205 extends distally from the distal end of the third region 190 and tapers distally to a larger diameter than the third region 190. The radiopaque tip 175 is welded to the distal end of this tapered region 205 of the body member 170.

When vibrational energy is transmitted distally along the waveguide 135, the amplitude of the vibrational energy increases due to the decreasing diameter of the waveguide 135 in the distal direction. The third region 190 can be sized to buckle and thus vibrate in a transverse mode when vibrational energy of a predetermined frequency and amplitude is transmitted thereto. The third region 190 of the waveguide 135 can, for example, be configured to produce multiple transverse nodes and anti-nodes along its length in response to the longitudinal vibrations transmitted along the waveguide 135.

As shown in FIG. 1, the coiled section 125 of the catheter 105 is disposed between and thermally bonded to the proximal and distal sections 115, 120 of the catheter 105. The coiled section 125 includes a helical-shaped tubular member 210 that defines a waveguide lumen 215 that extends along the longitudinal axis of the catheter 105. The helical-shaped tubular member 210 surrounds the third region 190 (FIG. 2) of the waveguide 135 with few contact points, which helps to reduce friction between the waveguide 135 and the coiled section 125 as the third region 190 of the waveguide 135 vibrates transversely during use. As a result, the dampening effect on the waveguide 135 can be reduced and the energy output of the waveguide 135 can be increased.

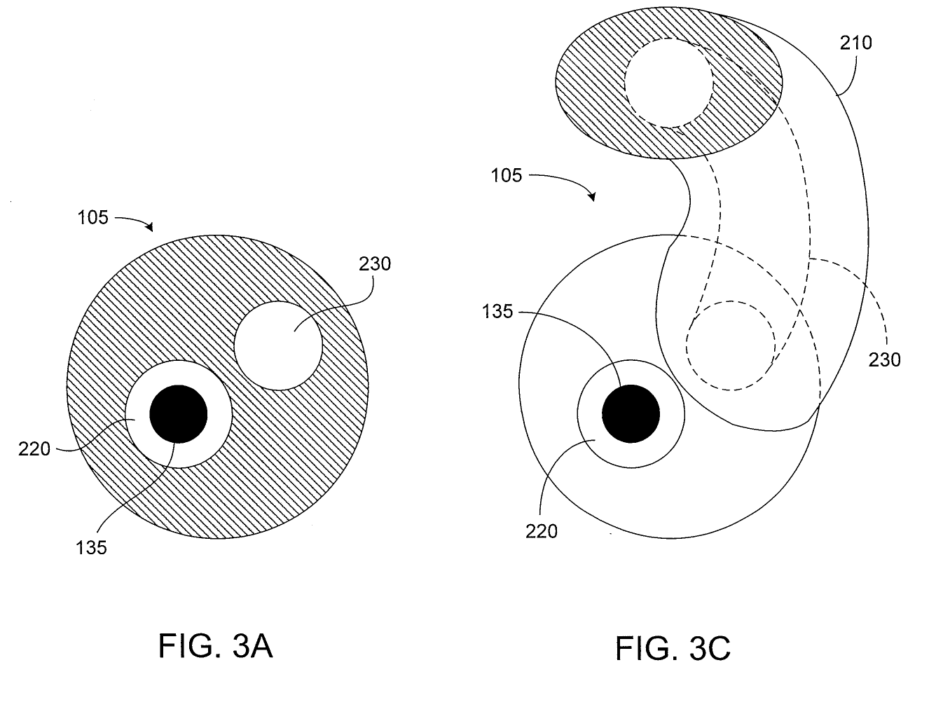

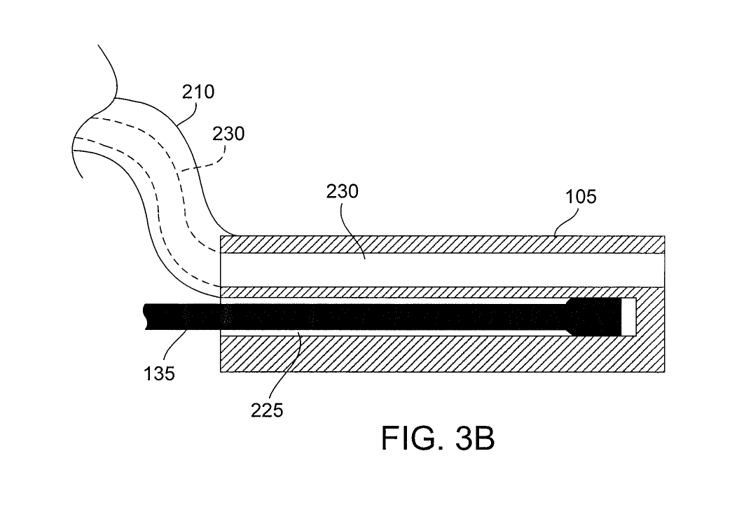

Referring to FIGS. 1 and 3A, the proximal section 115 of the catheter 105 defines a waveguide lumen 220 that is sized to receive the waveguide 135 therein. The waveguide lumen 220 extends from the proximal end of the proximal section 115 to the distal end of the proximal section 115. Referring to FIG. 3B, the distal section 120 of the catheter 105 defines a waveguide cavity 225 that is configured to receive the distal end portion of the waveguide 135. The length of the waveguide cavity 225 is generally sufficient to ensure that the distal end of the waveguide 135 remains disposed within the waveguide cavity 225 when the coiled section 125 of the catheter 105 is in a fully contracted position. The waveguide cavity 225 can, for example, have a length of about 0.5 centimeter to about 10 centimeters (e.g., about 1.0 centimeter to about 5.0 centimeters, about 2.0 centimeters). Maintaining the distal end of the waveguide 135 within the waveguide cavity 225 during use can help to ensure that the distal end of the waveguide 135 does not extend laterally through a helical opening 240 that extends along the coiled section 125. In certain embodiments, the catheter 105 includes a projection (e.g., an annular projection) that extends radially inward from the inner surface of the catheter near the proximal end of the waveguide cavity 225. This projection can further help to ensure that the distal end of the waveguide 135 does not pop out of the waveguide cavity 225 during use.

As shown in FIGS. 3A-3C, the catheter 105 also includes a guidewire lumen 230 that is sized to receive a guidewire therein. The guidewire lumen 230 extends from the proximal end of the catheter 105 to a distal end of the catheter 105, through each of the proximal, coiled, and distal sections 115, 125, 120.

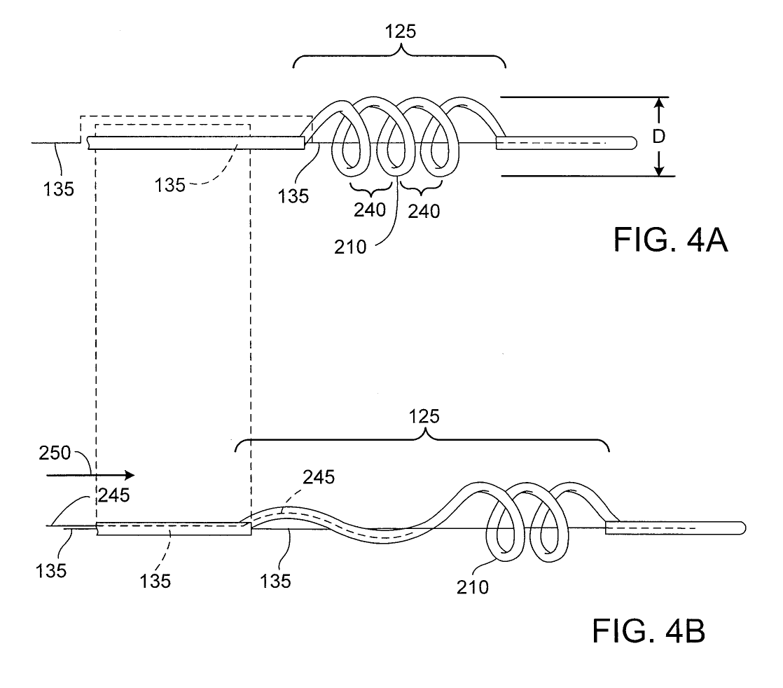

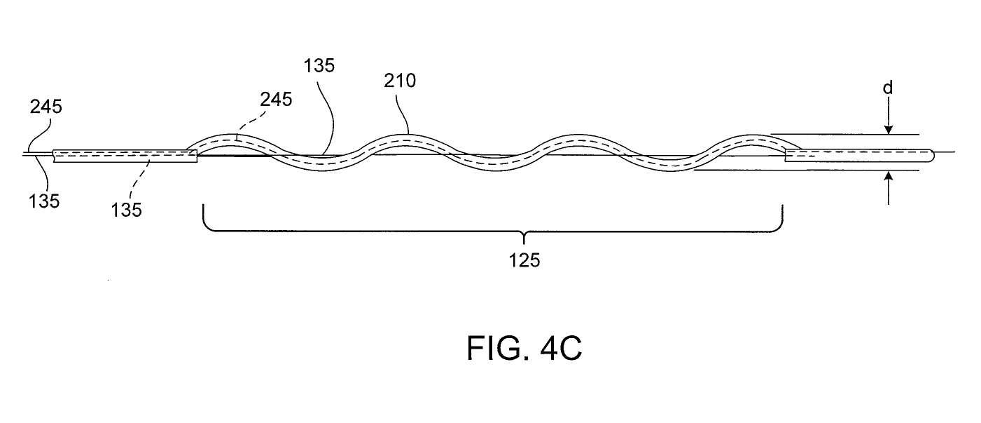

Referring to FIGS. 4A-4C, the coiled section 125 of the catheter 105 is radially displaceable, relative to the waveguide 135, between the radially expanded position (shown in FIG. 4A) and a radially contracted position (shown in FIG. 4C). Referring to FIG. 4A, the helical-shaped tubular member 210 of the coiled section 125 is resiliently biased toward the radially expanded position, and, as a result, when no guidewire is disposed in the portion of the guidewire lumen 230 running through the helical-shaped tubular member 210 of the coiled section 125, the coiled section 125 expands radially outward from the waveguide 135. In the radially expanded position, the coiled section 125 can have a diameter D of about 2.0 millimeters to about 12 millimeters and a length of about 20 millimeters to about 120 millimeters. The helical-shaped tubular member 210 forms the helical opening 240 that extends about the waveguide 135 and allows for the release of vibrational energy from the waveguide 135 to the surrounding environment. Because the opening 240 extends helically about the waveguide 135, it permits the release of vibrational energy in multiple radial directions (e.g., in all radial directions about the 360 degree circumference of the waveguide 135) without substantial rotation of the catheter 105.

Referring to FIG. 4B, as a relatively stiff, stainless steel guidewire 245 is advanced (as indicated by arrow 250) into the portion of the guidewire lumen 230 extending though the helical-shaped tubular member 210 of the coiled section 125, the helical-shaped tubular member 210 lengthens and is radially displaced towards the waveguide 135. To dispose the guidewire 245 within the portion of the guidewire lumen 230 extending though the helical-shaped tubular member 210, the guidewire is inserted (via an opening formed in the sidewall of the catheter in the proximal section 115) into the portion of the guidewire lumen 230 extending through the proximal section 115 of the catheter 105 and advanced distally until a desired portion of the guidewire 245 is positioned within the portion of the guidewire lumen 230 extending though the helical-shaped tubular member 210. The guidewire 245 includes a distal section that is more flexible than the rest of the guidewire. The distal section can, for example, have a smaller diameter and/or be formed of a more flexible material than the rest of the guidewire. Due to the increased flexibility of the distal section of the guidewire 245, the distal section of the guidewire 245 can be passed through the helical-shaped tubular member 210 of the coiled section 125 relatively easily. For example, the flexibility of the distal section of the guidewire 245 can permit the distal section of the guidewire 245 to track the helical portion of the guidewire lumen 230 extending through the helical-shaped tubular member 210, causing the helical-shaped tubular member 210 of the coiled section 125 to partially contract. Due to the partial contraction of the helical-shaped tubular member 210 of the coiled section 125, the relatively stiff proximal section of the guidewire 245 can then more easily be passed through the helical-shaped tubular member 210 of the coiled section 125, causing the helical-shaped tubular member 210 of the coiled section 125 to be fully contracted.

As shown in FIG. 4C, when the guidewire 245 is disposed in a position extending all the way through the coiled section 125 such that the relatively stiff proximal portion of waveguide is disposed in the helical-shaped tubular member 210 of the coiled section 125, the helical-shaped tubular member 210 is maintained in a contracted position. In this contracted position, the coiled section 125 can have a diameter d about 0.5 millimeters to about 4.0 millimeters and a length of about 20 millimeters to about 120 millimeters.

The helical-shaped tubular member 210 of the coiled section 125 of the catheter typically has a smaller diameter than the proximal and distal sections 115, 120 of the catheter 105. The helical-shaped tubular member 210 can have an outer diameter of about 0.5 millimeter to about 3.0 millimeters and/or an inner diameter of about 0.25 millimeter to about 2.7 millimeters. The proximal section 115 of the catheter 105 can have an outer diameter of about 0.7 millimeter to about 3.3 millimeters and/or an inner diameter of about 0.5 millimeter to about 3.0 millimeters. The distal section 120 of the catheter 105 can have an outer diameter of about 0.5 millimeter to about 3.0 millimeters and/or one or more inner diameters of about 01.5 millimeters to about 2.7 millimeters. While exemplary dimensions of the catheter have been provided above, it should be noted that the dimensions of the various sections of the catheter 105 will depend upon the intended use of the catheter. Catheters to be used within neurological blood vessels, for example, would generally be smaller than catheters to be used within peripheral blood vessels.

In certain embodiments, the helical-shaped tubular member 210 of the coiled section 125, the proximal section 115, and the distal section 120 are formed of one or more thermoset polymers, such as elasatomers (e.g., rubbers), fluoropolymers, methacrylates, polyesters, polyimides, polyurethanes, silicones, and combinations of these materials. Alternatively or additionally, the helical-shaped tubular member 210 of the coiled section 125, the proximal section 115, and the distal section 120 can include one or more thermoplastic polymers, such as polyether-block co-polyamide polymers (e.g., PEBAX®), nylons, polyethylenes, polyurethanes, and combinations of these materials. In certain embodiments, each of the helical-shaped tubular member 210 of the coiled section 125, the proximal section 115, and the distal section 120 are formed of polyether-block co-polyamide polymers (e.g., PEBAX®).

In some embodiments, the proximal and distal sections 115, 120 are formed of a different type or different grade of polymer than the helical-shaped tubular member 210 of the coiled section 125. In such embodiments, for example, the proximal and distal sections 115, 120 can be formed of polyether-block co-polyamide polymers (e.g., PEBAX®), copolyester elastomers (e.g., Arnitel® copolyester elastomers), thermoplastic polyester elastomers (e.g., Hytrel®), thermoplastic polyurethane elastomers (e.g., Pellethane™), polyeolefins (e.g., Marlex® polyethylene, Marlex® polypropylene), HDPEs, low-density polyethylenes (LDPEs), polyamides (e.g., Vestamid®), polyetherether ketones (e.g., PEEK™), or combinations of these materials.

In certain embodiments, the proximal and distal sections 115, 120 are formed of different materials. The distal section 120 can, for example, be formed of a material that is less rigid than the material of the proximal section 115. In some embodiments, for example, the distal section 120 is formed of PEBAX® 55D and the proximal section 115 is formed of PEBAX® 72D. The helical-shaped tubular member 210 of the coiled section 125 can be formed of a material that has an intermediate stiffness as compared to the proximal and distal sections 115, 120. In some embodiments, for example, the distal section 120 is formed of PEBAX® 55D, the helical-shaped tubular member 210 of the coiled section 125 is formed of PEBAX® 63D, and the proximal section 115 is formed of PEBAX® 72D.

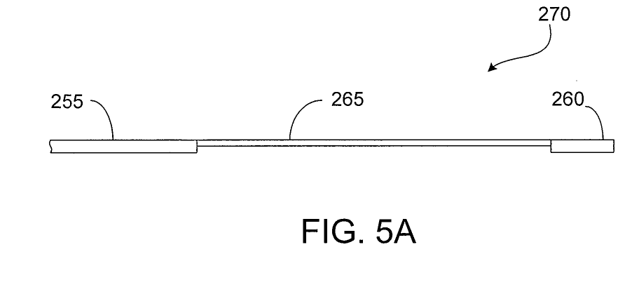

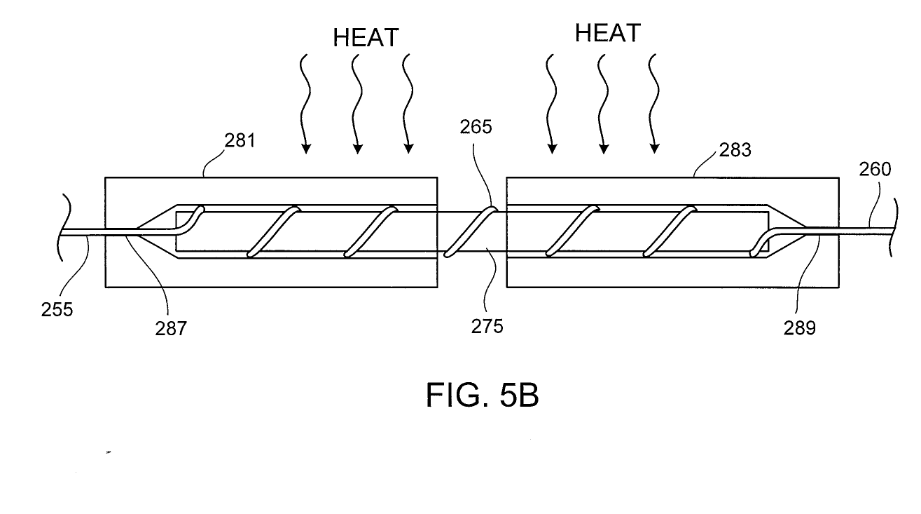

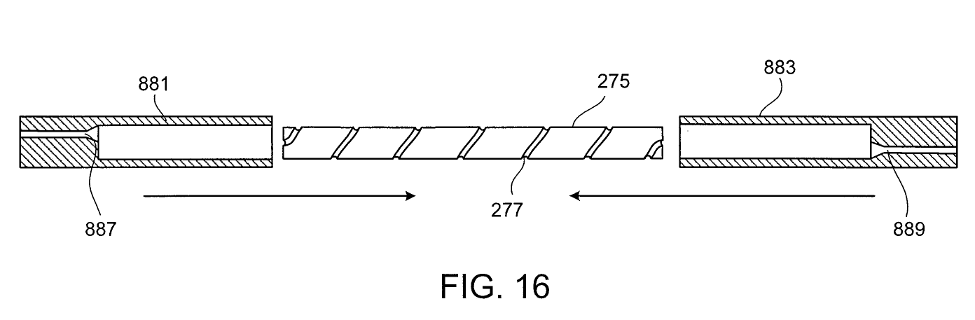

FIGS. 5A and 5B illustrate a method of making the catheter 105. As shown in FIG. 5A, first and second tubes 255, 260 are thermally bonded to opposite ends of a third, smaller diameter tube 265 to form a tube assembly 270. Referring to FIG. 5B, the third tube 265 of the tube assembly 270 is then wrapped around a mandrel 275 and held in a coiled configuration by sleeves 281, 283 that surround opposite end portions of the mandrel 275 and the third tube 265. The mandrel 275 also includes a helical groove 277 (shown in FIG. 16) extending along its length. The third tube 265 is partially disposed in the groove 277 to help maintain the third tube 265 in the coiled configuration. The first and second tubes 255, 260 extend through bores 287, 289 extending through end regions of the sleeves 281, 283. As a result, the first and second tubes 255, 260 are held in a substantially straight (e.g., uncoiled) configuration.

While holding the third tube 265 in the coiled configuration, the material of the third tube 265 is heated to a temperature between body temperature and the glass transition temperature of the material. In some embodiments, for example, the third tube 265 is heated for about 10 minutes to about 30 minutes at a temperature of about 60 degrees Celsius to about 80 degrees Celsius. The temperature and time used to heat the third tube 265, however, will vary among the various different materials that can be used to form the third tube 265. In addition, as the temperature to which the third tube 265 is heated increases, the amount of time for which the third tube 265 is heated can decrease, and vice versa.

To heat the third tube 265, the entire tube assembly 270 and the mandrel 275 can be placed in a furnace and heated to the desired temperature. Alternatively or additionally, other heating techniques can be used. For example, the third tube 265 can be heated in an oven, in hot water, by infrared energy, and/or by radiant heat. Similarly, with any of these heating techniques, the heat can be applied to substantially only the third tube 265 and not the entire tube assembly 270. As a result of the heat applied to the third tube 265, the material of the third tube 265 becomes set in the coiled configuration.

After allowing the third tube 265 to cool in the coiled configuration, the tube assembly 270 is removed from the mandrel 275 by pulling the sleeves 281, 283 away from one another and off of the mandrel 275 and then sliding the coiled third tube 265 off of the mandrel 275. The tube assembly 270 can then be subjected to further processing steps to complete the catheter 105. The first, second, and third tubes 255, 260, 265 of the tube assembly 270 become the proximal, distal, and coiled sections 115, 120, 125, respectively, of the resulting catheter 105.

While the third tube 265 has been described as being held in the coiled configuration by the combination of the sleeves 281, 283 and the helical groove 277 in the mandrel 275, other techniques can alternatively or additionally be used. In some embodiments, for example, the first and second tubes 255, 260 are fixed (e.g., clamped) to the mandrel 275 to maintain the third tube 265 in a coiled configuration around the mandrel 275.

While the coiled section 125 of the catheter 105 has been described as being formed by wrapping the tube assembly 270 around the mandrel 275 and heating the tube assembly 270, other methods can alternatively or additionally be used to form the coiled section 125. In some embodiments, for example, the tube assembly 270 is disposed within the lumen of a slightly larger metal tube that has a coil-shaped portion. The tube assembly 270 is passed through the metal tube until the third tube 265 of the tube assembly 270 is disposed within the coil-shaped portion of the larger tube, which imparts a coil shape to the third tube 265. In this configuration, the third tube 265 and the coil-shaped portion of the metal tube in which the third tube 265 is disposed are heated (e.g., by using one or more of the heating techniques described above). After allowing the third tube 265 to cool, the tube assembly 270 is removed from the lumen of the metal tube. The tube assembly 270 can, for example, simply be pulled through the lumen of the metal tube. As a result of the heat applied to the third tube 265, the material of the third tube 265 remains set in a coiled configuration after being removed from the metal tube. After removing the tube assembly 270 from the metal tube, the tube assembly 270 can undergo further processing to complete the catheter 105.

While the coil-shaped tubular mold described above has been described as being formed of metal, the tubular mold can alternatively be formed of one or more other materials that has a higher melting temperature than the third tube 265 of the tube assembly 270. For example, metal alloys and/or polymers with high melting temperatures can be used.

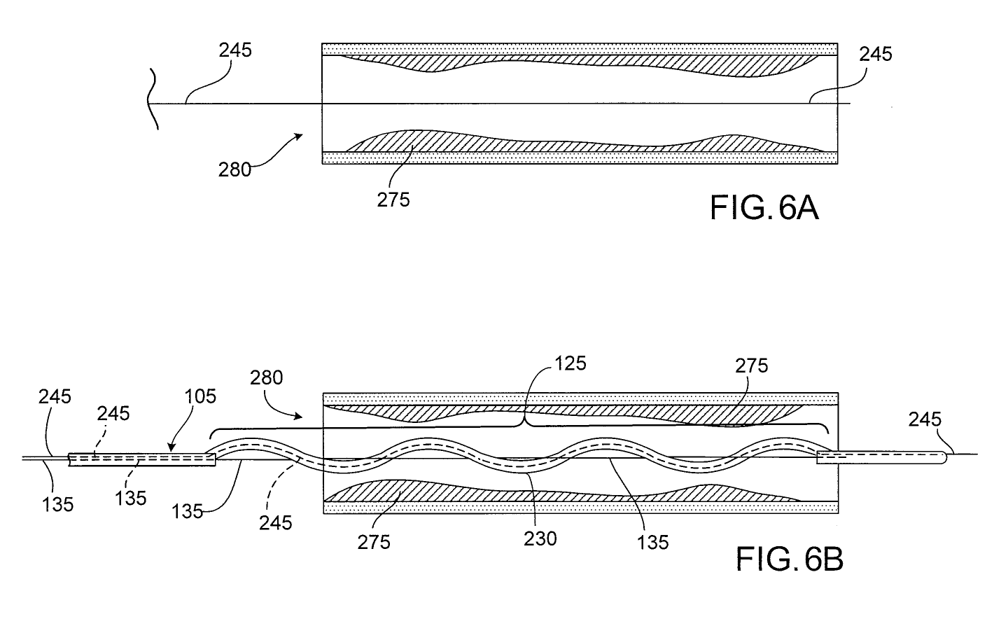

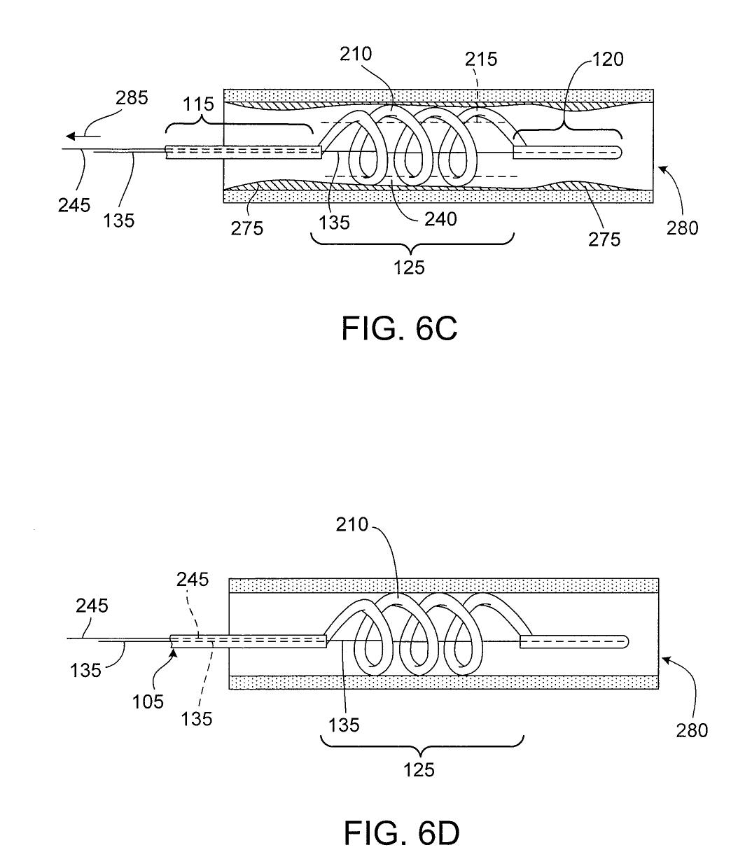

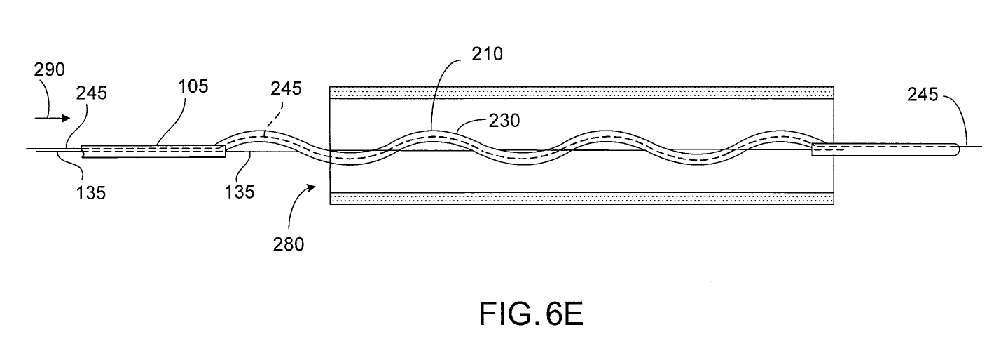

FIGS. 6A-6F diagrammatically illustrate a method of using the medical system 100 to ablate a thrombus 275 within a blood vessel 280 of a patient. As shown in FIG. 6A, after threading the guidewire 245 through the catheter 105 (from the proximal end of the catheter 105 to the distal end of the catheter 105), the distal section of the guidewire 245, which extends distally to the distal end of the catheter 105, is introduced into the blood vessel 280 and navigated to a region of the blood vessel 280 including the thrombus 275. As the distal section of the guidewire 245 is navigated through the blood vessel 280, the proximal section of the guidewire 245 (not shown in FIG. 6A) is disposed within the catheter 105, maintaining the coiled section 125 of the catheter in the contracted position. The guidewire 245 exits the catheter 105 via the opening formed in the side wall of the proximal section 115 of the catheter. This allows the user to grasp the exposed proximal end portion of the guidewire 245 while threading the guidewire 245 through the blood vessel 280.

Referring to FIG. 6B, a distal portion of the medical system 100 is then inserted into the blood vessel 280 by sliding the catheter 105 over the guidewire 245. With the guidewire 245 disposed in the guidewire lumen 230, the coiled section 125 of the catheter 105 is held in a contracted position, as discussed above. The catheter 105 and the waveguide 135 are advanced as a unit along the guidewire 245 toward the thrombus 275 in the blood vessel 280. The catheter 105 and the waveguide 135 are navigated through the blood vessel 280 until the contracted coiled section 125 of the catheter 105 and the portion of the waveguide 135 exposed by the helical opening 240 in the contracted coiled section 125 are disposed within the thrombus 275. The catheter 105 and the waveguide 135 can be guided through the blood vessel by feel or using an image guidance technique, such as fluoroscopy or X-ray. In those cases in which an imaging guidance technique is used, the catheter 105 and/or the waveguide 135 can, for example, include radiopaque markers that can be seen by using the desired imaging equipment to help the user position the system as desired within the blood vessel 280.

Referring to FIG. 6C, after positioning the contracted coiled section 125 of the catheter 105 and the portion of the waveguide 135 exposed by the helical opening 240 of the contracted coiled section 125 within the thrombus 275, the guidewire 245 is proximally retracted into the proximal section 115 of the catheter 105 (as indicated by arrow 285). As a result, the helical-shaped tubular member 210 of the coiled section 125 expands into contact with the inner surface of the thrombus 275. The helical-shaped tubular member 210 of the coiled section 125 is malleable and thus generally conforms to the size and shape of the thrombus 275. The expansion of the helical-shaped tubular member 210 helps to center the waveguide 135 within the blood vessel 280.

As an alternative to fully retracting the guidewire 245 into the proximal section 115 of the catheter 105, the guidewire 245 can be retracted so that the flexible distal section of the guidewire 245 is disposed in the helical-shaped tubular member 210, allowing the helical-shaped tubular member 210 to only partially expand.

After positioning the catheter 105 and the waveguide 135 as desired within the blood vessel 280, an electrical energy source is activated to deliver electrical energy to the acoustic assembly 130 (shown in FIG. 1), causing the transducer rings 155, 160 and the horn 145 of the acoustic assembly 130 to vibrate at a frequency of about 40 kHz. Due to the vibrational energy transmitted to the waveguide 135 by the acoustic assembly 130, the portion of the waveguide 135 disposed within the coiled section 125 of the catheter 105 vibrates transversely and thus delivers vibrational energy radially to the thrombus 275 via the helical opening 240 of the coiled section 125. This vibrational energy serves to ablate the thrombus 275 (e.g., by causing cavitation in fluid within or adjacent the thrombus 275). The helical-shaped tubular member 210 of the coiled section 125 inhibits the waveguide 135 from directly contacting the wall of the blood vessel 280 but allows the waveguide 135 to freely move transversely within the axial lumen 215 extending through the coiled section 125. The waveguide 135 can be vibrated until the thrombus 275 has been substantially entirely ablated, as shown in FIG. 6D.

Referring to FIG. 6E, after ablating the thrombus to a desired degree, the guidewire 245 is advanced (as indicated by arrow 290) into the portion of the guidewire lumen 230 that extends through the helical-shaped tubular member 210 of the coiled section 125 of the catheter 105. As a result, the helical-shaped tubular member 210 of the coiled section 125 is radially displaced toward the waveguide 135 and into its radially contracted position. The catheter assembly 100 is then withdrawn from the blood vessel 280.

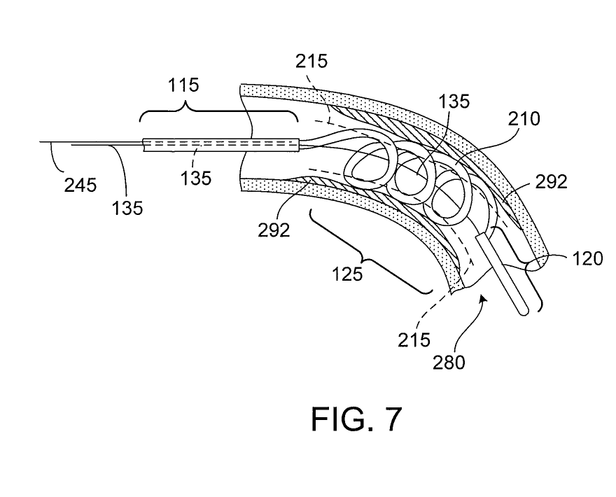

While the system 100 has been illustrated as being used to ablate a thrombus within a relatively straight portion of a blood vessel, the system 100 can also be used to ablate thrombi within tortuous regions of blood vessels. As shown in FIG. 7, for example, the system 100 is shown disposed adjacent a thrombus 292 within a tortuous region of the blood vessel 280. The coiled section 125 of the catheter 105 is in the radially expanded state such that the helical-shaped tubular member 210 of the coiled section 125 creates a radial boundary about the waveguide 135 and thus inhibits the waveguide 135 from contacting the wall of the blood vessel 280. In addition, the radial boundary created by the helical-shaped tubular member 210 inhibits the distal portion of the waveguide 135 from popping out of the waveguide cavity 225 (shown in FIG. 3B) formed in the distal section 120 of the catheter 105. In particular, because the helical-shaped tubular member 210 limits the extent to which the waveguide 135 can move radially within the coiled section 125, it also limits the extent to which the distal portion of the waveguide 135 (e.g., the portion of the waveguide 135 disposed within the distal section 120 of the catheter 105) can move axially in the proximal direction.

While the guidewire 245 has been described as having a flexible distal section, the guidewire 245 can alternatively have a flexible proximal section. In such cases, rather than inserting the distal end of the guidewire 245 into the proximal opening of the guidewire lumen 230 and advancing the guidewire distally until it exits the distal opening of the guidewire lumen, the proximal end of the guidewire 245 is inserted into the distal opening of the guidewire lumen 230 and advanced proximally through the catheter 105 until it exits the proximal opening of the guidewire lumen 230. Thus, in such cases, the guidewire 245 can be positioned within the blood vessel prior to loading the catheter 105 onto the guidewire 245.

In some embodiments, the guidewire 245 includes flexible proximal and distal sections with a relatively stiff intermediate section therebetween. In such embodiments, the guidewire 245 can be loaded into the catheter 105 via either the proximal or distal opening of the guidewire lumen 230.

While the guidewire 245 has been described as a stainless steel guidewire, other types of guidewires can alternatively or additionally be used. For example, guidewires formed of various other metals and/or rigid polymers can alternatively or additionally be used. In some embodiments, a variable stiffness guidewire assembly is used. Such a variable stiffness guidewire assembly can include a relatively, flexible sheath that surrounds a relatively rigid guidewire. During use, the distal portion of the guidewire assembly can be advanced into a thrombus. The catheter 105 can then be threaded over the guidewire assembly such that a portion of the guidewire assembly is disposed within the portion of the guidewire lumen 230 extending through the helical-shaped tubular member 210 of the coiled section 125 of the catheter 105 and forces the coiled section 125 of the catheter 105 into the radially contracted position. In order to allow the coiled section 125 to expand radially, the relatively rigid guidewire of the guidewire assembly can be retracted while leaving the distal portion of the flexible sheath inserted within the thrombus. Because the distal portion of the flexible sheath remains within the thrombus during the procedure, the guidewire can be easily tracked back into the occlusion via the sheath if desired.

While the transducer rings 155, 160 and the horn 145 of the acoustic assembly 130 have been described as vibrating at a frequency of about 40 kHz, they can vibrate at higher or lower frequencies. In general, they vibrate at a frequency of at least about 10 kHz (e.g., at least about 15 kHz, at least about 20 kHz, at least about 30 kHz) and/or at most about 100 kHz (e.g., at most about 90 kHz, at most about 80 kHz, at most about 70 kHz). In some embodiments, they vibrate at a frequency of about 10 kHz to about 100 kHz (e.g., from about 15 kHz to about 90 kHz, from about 20 kHz to about 80 kHz, from about 30 kHz to about 70 kHz, from about 35 kHz to about 45 kHz, from about 37 kHz to about 43 kHz, from about 39 kHz to about 41 kHz).

While the waveguide 135 has been described as being releasably secured to the horn 145 of the acoustic assembly 130 by the coupler 165, which is crimped to the proximal end portion of the waveguide 135 and screwed into or onto the horn, other techniques can be used to secure the waveguide 135 to the horn 145. A coupler can, for example, be welded to the proximal end portion of the waveguide 135 and/or to a distal portion of the horn 145.

As an alternative to the acoustic assembly 130 described above, any of various other types of acoustic assemblies can be used. In some embodiments, for example, an acoustic assembly that utilizes other types of piezoelectric elements or magnetorestrictive elements can be used to vibrate the waveguide.

While certain embodiments have been described above, other embodiments are possible.

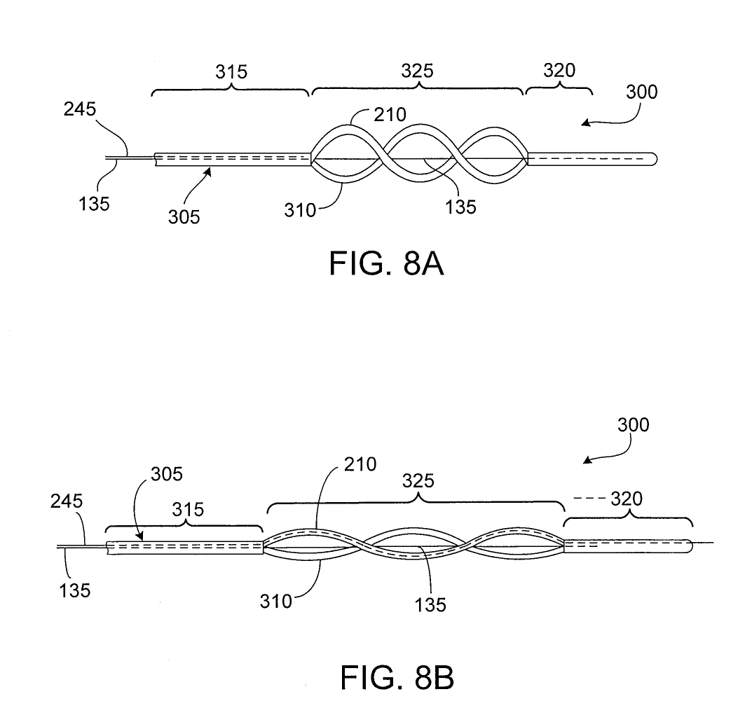

While the coiled section 125 of the catheter 105 has been described as including only a single helical-shaped tubular member 210, in some embodiments, the coiled section of the catheter includes multiple (e.g., two, three, four, five, or more) helical-shaped tubular members. For example, FIGS. 8A and 8B illustrate a medical system 300 that includes a catheter 305 with a coiled section 325 that includes a pair of helical-shaped tubular members 210, 310 wound about the waveguide 135. As illustrated in FIG. 8B, a single guidewire 245 can be used to radially contract or straighten both of the helical-shaped tubular members 210, 310. As the guidewire 245 is advanced within the lumen of the helical-shaped tubular member 210, a proximal section 315 and a distal section 320 of the catheter 305 move axially away from one another, causing the helical-shaped tubular member 310 to lengthen and radially contract. The system 300 illustrated in FIGS. 8A and 8B can be used in a manner similar to the system 100 described above. Due to the increased number of helical-shaped tubular members in the coiled section 325 of the catheter 305, the coiled section 325 can provide a greater number of contact points with the waveguide 135 and thus further inhibit the waveguide 135 from contacting a blood vessel wall during use.

While the coiled section 325 of the catheter 305 has been described as including multiple helical-shaped tubular members 210, 310, in some embodiments, only one of these members is tubular. For example, one of the members can be a helical-shaped tubular member in which the guidewire 245 is received and the remaining member(s) can be helical-shaped solid members, such as thin rods. The helical shaped solid members can be formed of any of the various materials described herein with respect to the helical-shaped tubular member 210. Alternatively or additionally, other materials can be used to form the helical-shaped solid members. In some embodiments, for example, the helical-shaped solid members are formed of polymer coated wires.

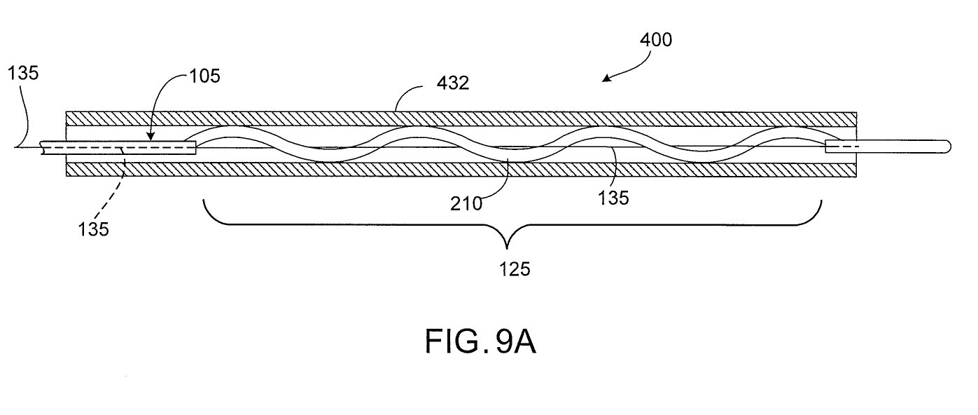

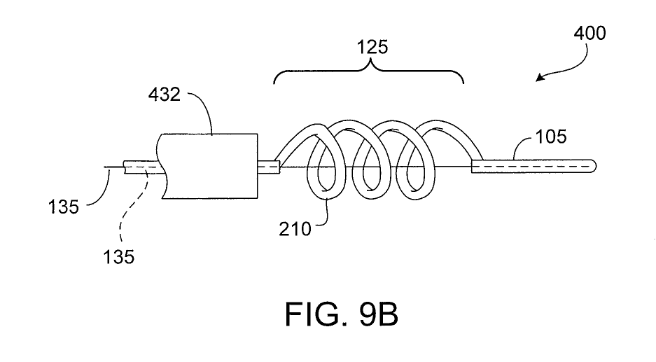

While the coiled sections of the catheters discussed above have been described as being radially displaced by axially advancing or retracting a guidewire within a lumen of one of the members that form the coiled section, expansion of the coiled section can be controlled in other ways. For example, FIGS. 9A and 9B illustrate a medical system 400 that includes an outer sheath 432 that at least partially surrounds the catheter 105. The outer sheath 432 is axially displaceable relative to the catheter 105. The outer sheath 432 can be retracted from a first position (shown in FIG. 9A) in which the outer sheath 432 is disposed over the coiled section 125 of the catheter 105, thereby maintaining the helical-shaped tubular member 210 of the coiled section 125 in a radially compressed or contracted state, to a second position (shown in FIG. 9B) in which the outer sheath 432 is retracted to expose the coiled section 125, thereby allowing the helical-shaped tubular member 210 of the coiled section 125 to expand. In use, a distal portion of the medical system 400 is advanced into and navigated through a blood vessel with the outer sheath 432 surrounding the coiled section 125 of the catheter 105. This can be done with or without the aid of a guidewire. The catheter 105, the outer sheath 432, and the waveguide 135 are advanced as a unit through the blood vessel until the radially compressed coiled section 125 of the catheter 105 is located adjacent a thrombus within the blood vessel. The outer sheath 432 is then retracted relative to the catheter 105 toward the second position, allowing the helical-shaped tubular member 210 of the coiled section 125 to expand into contact with the thrombus and/or the wall of the blood vessel. The thrombus is then ablated in the manner described above. After ablating the thrombus, the outer sheath 432 is axially displaced relative to the catheter 105 back toward the first position to radially compress the helical-shaped tubular member 210 of the coiled section 125. The catheter 105, the outer sheath 432, and the waveguide 135 are then withdrawn from the blood vessel.

An outer sheath can similarly be used in combination with any of the various other catheters described herein to aid in radially displacing a section of the catheter.

Other techniques can also be used to radially displace coiled sections of catheters.

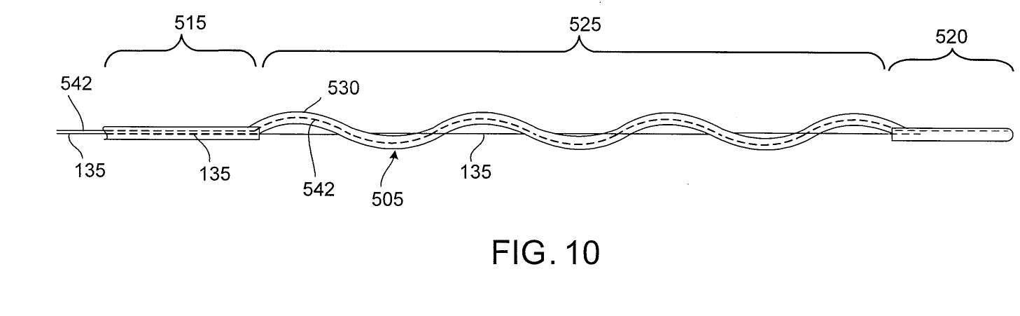

FIG. 10, for example, illustrates a medical system 500 that includes a catheter 505 that is used in conjunction with a guidewire 542 formed of a nickel-titanium shape memory alloy, such as nitinol. The catheter 505 includes a proximal section 515, a distal section 520, and a flexible, intermediate section 525 disposed between and attached to the proximal and distal sections 515, 520. The intermediate section 525 can be formed of any of the various materials described herein with regard to helical-shaped tubular member 210. The intermediate section 525 is wound about the waveguide 135. The proximal and distal sections 515, 520 can be formed of any of the various materials described above with regard to the proximal and distal sections 115, 120 of catheter 105.

The intermediate section 525 of the catheter 505 can have dimensions similar to those of the helical-shaped tubular member 210 of catheter 105 discussed above. Similarly, the proximal and distal sections 515, 520 can have dimensions similar to those of the proximal and distal sections 115, 120 of catheter 105 discussed above.

The proximal, intermediate, and distal sections 515, 525, 520 together define a lumen 530 that extends from the proximal end of the catheter 505 to the distal end of the catheter 505. The guidewire 542 is disposed within the lumen 530 during use. Similar to the systems described above, the proximal and distal sections 515 and 520 of the catheter 505 also include a lumen in which the waveguide 135 is disposed. The guidewire 542 is constructed to change shape when heated above a predetermined temperature. Thus, as discussed below, radial displacement of the intermediate section 525 of the catheter 505 can be controlled by controlling the temperature of the guidewire 542.

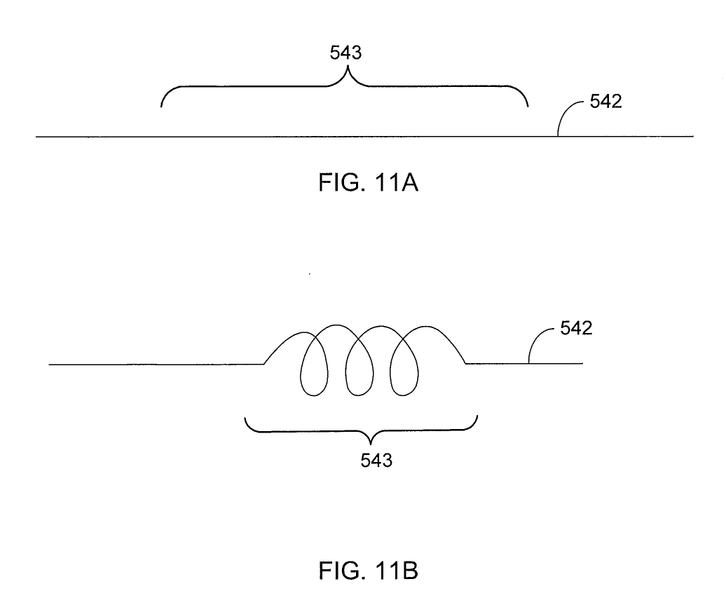

As illustrated in FIG. 11 A, the guidewire 542 has a low-temperature shape that is substantially straight along its length. Referring to FIG. 11 B, the guidewire 542 has a high-temperature shape in which a deformable section 543 of the guidewire 542 forms a coil.

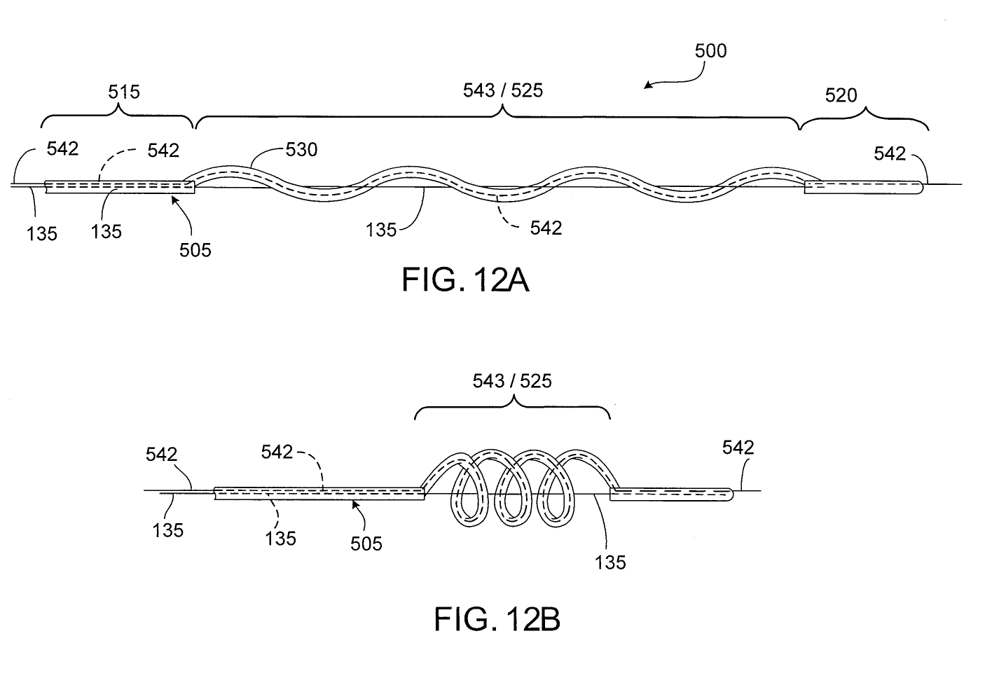

Referring to FIG. 12A, the guidewire 542 is disposed within the guidewire lumen 530 of the catheter 505 and is positioned such that the deformable section 543 of the guidewire 542 is disposed within the intermediate section 525 of the catheter 505. The deformable section 543 of the guidewire 542 and the intermediate section 525 of the catheter 505 can, for example, be situated in a desired position relative to one another by providing radiopaque markers on both the intermediate section 525 of the catheter 505 and on the deformable section 543 of the guidewire 542. The radiopaque markers can be arranged so that they align with one another when the deformable section 543 of the guidewire 542 is disposed within the intermediate section 525 of the catheter 505. Thus, by using an imaging technique, such as fluoroscopy or X-ray, the user can determine when the catheter 505 has been advanced within the blood vessel (over the guidewire 542) to an extent such that the deformable section 543 of the guidewire 542 is disposed within the intermediate section 525 of the catheter 505.

Referring to FIG. 12B, when the deformable section 543 of the guidewire 542 is disposed within the intermediate section 525 of the catheter 505, the guidewire 542 can be heated (e.g., by delivering a heated fluid to the guidewire 542 via the guidewire lumen 530 of the catheter 505 or by applying an electrical current to a proximal end portion of the guidewire 542) to cause the guidewire 542 to assume its high-temperature shape, thereby imparting a coiled shape to the intermediate section 525 of the catheter 505.

During use, the guidewire 542 is advanced in its low-temperature shape into a blood vessel of a patient toward a thrombus within the blood vessel. Next the catheter 505 is advanced over the guidewire 542 in a manner such that the guidewire 542 extends within the guidewire lumen 530 of the catheter 505. The catheter 505 and the waveguide 135 can be guided through the blood vessel by feel or using an image guidance technique, such as fluoroscopy or X-ray. The catheter 505 is positioned at a treatment site such that the deformable section 543 of the guidewire 542 is disposed within the intermediate section 525 of the catheter 505. Next, the guidewire 542 is heated to impart an expanded, coiled shape to the intermediate section 525 of the catheter 505. The thrombus is then ablated by vibrating the waveguide 135 within the coiled section of the catheter 505, as discussed above. Subsequently, the deformable section 543 of the guidewire 542 is radially displaced by cooling the guidewire 542 such that the guidewire 542 assumes its low-temperature shape. The catheter 505 and the waveguide 135 are then withdrawn from the blood vessel. The guidewire 542 can be simultaneously removed from the blood vessel with the catheter 505 and waveguide 135, or the guidewire 542 can be removed from the blood vessel after removing the catheter 505 and the waveguide 135.

While the deformable section 543 of the guidewire 542 has been described as being in a coiled shape when heated and being in a straight configuration when cooled, the guidewire 542 can alternatively be configured so that the deformable section 543 is in a straight configuration when heated and is in a coiled shape when cooled. In such cases, a cooled fluid can be delivered to the deformable section 543 during use to impart a coiled shape to the intermediate section 525 of the catheter 505.

While the intermediate section 525 of the catheter 505 has been described as being wrapped around the waveguide 135, other configurations are possible. In some embodiments, for example, the intermediate section 525 extends substantially linearly between the proximal and distal sections 515, 520 of the catheter 505. In such embodiments, the waveguide 135 is axially displaceable relative to the catheter 505. During use, the waveguide 135 can be contained within the proximal section 515 of the catheter 505 until the intermediate section 525 of the catheter 505 has been radially expanded to the coiled configuration. At that time, the waveguide 135 can be advanced distally into the central lumen now formed by the coiled intermediate section 525. The treatment can then be carried out in a manner similar to those treatments described above. After completion of the treatment, the waveguide 135 can be retracted into the proximal section 515 of the catheter 505 and the intermediate section 525 of the catheter 505 can be radially contracted by cooling the guidewire 542. After contracting the intermediate section 525 of the catheter 105, the catheter 505 and the waveguide 135 are withdrawn from the blood vessel.

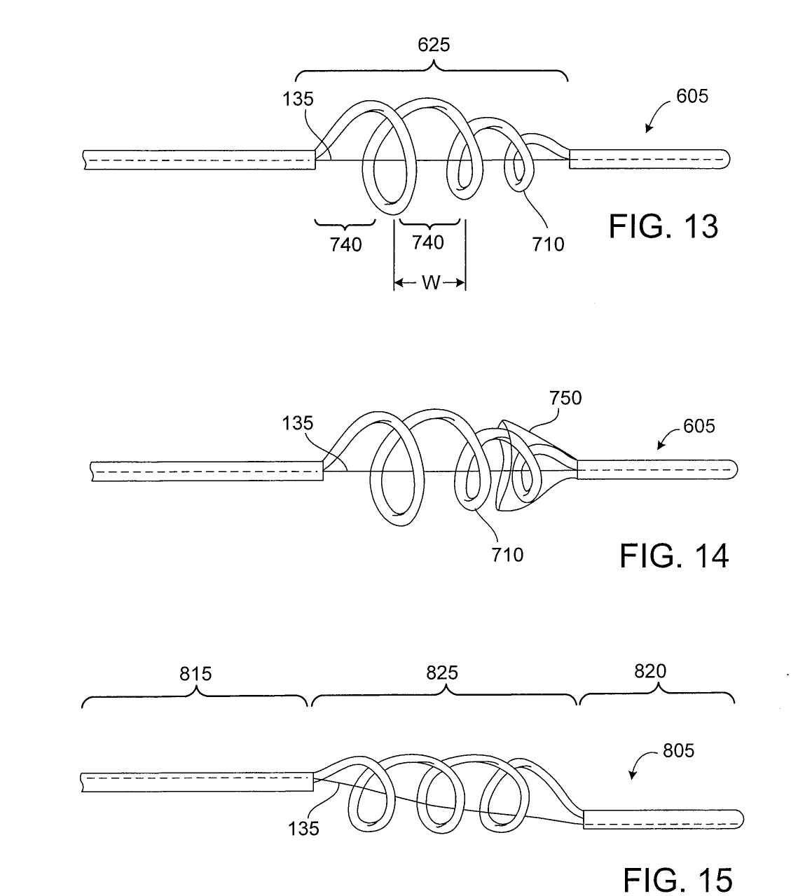

While the pitch and diameter of the coiled sections 125, 325 and the intermediate section 525 of the catheters above, when in the expanded state, are illustrated as being substantially constant, other configurations are possible. As shown in FIG. 13, for example, a catheter 605 has a coiled section 625 (shown in the expanded state) formed of a helical-shaped tubular member 710. The pitch and diameter of the helical-shaped tubular member 710 decrease from the proximal end of the coiled section 625 to the distal end of the coiled section 625. As a result, the width w of a helical opening 740 formed by the helical-shaped tubular member 710 gradually decreases from the proximal end of the coiled section 625 to the distal end of the coiled section 625. The wider portions of the helical opening 740 near the proximal end of the coiled section 625 allow a greater amount of energy emitted from the waveguide 135 to pass radially outward through the coiled section 625 than the narrower portions of the helical opening 740 near the distal end of the coiled section 625. In addition, the wider portions of the helical opening 740 allow a larger portion of the thrombus to fit within the helical opening 740 such that a larger portion of the thrombus can be located in close proximity to the waveguide 135. As a result of this arrangement, the portion of the thrombus surrounding the proximal end region of the coiled section 625 can be ablated faster than the portion of the thrombus surrounding the distal end region of the coiled section 625.

In addition to allowing vibrational energy to pass through the coiled section 625 and providing the waveguide 135 with closer access to portions of the thrombus, the helical opening 740 permits dislodged portions of the thrombus to enter the interior of the coiled section 625 where the dislodged portions can become trapped. For example, in situations where blood is flowing through the blood vessel toward the distal end of the coiled section 625, relatively large dislodged portions of the thrombus can pass through the wider portions of the helical opening 740 near the proximal end of the coiled section 625 and then travel distally toward the distal end of the coiled section 625 where they become trapped. Because the helical opening 740 becomes narrower near the distal end of the coiled section 625, some dislodged portions that were capable of passing though the proximal end region of the helical opening 740 will not be capable of passing through the distal end region of the helical opening 740 and will thus become trapped inside the coiled section 625. As the waveguide 135 continues to vibrate, the trapped portions of the thrombus will be ablated until they are small enough to pass through the distal end region of the helical opening 740 and back into the main bloodstream.

In some embodiments, a portion of the coiled section 625 is surrounded by a film. This arrangement can assist in trapping dislodged portions of the thrombus. As shown in FIG. 14, for example, a polymeric film (e.g., a polyurethane film) 750 surrounds and is secured to the distal end portion of the coiled section 625 of the catheter 605. The film 750 prevents even those dislodged portions of the thrombus that are small enough to pass through the distal end regions of the helical opening 740 from passing into the main bloodstream. Thus, even relatively small portions of the dislodged thrombus can be contained within the coiled section 625. These dislodged portions can be removed from the blood vessel along with the catheter 605 after treatment.

In certain embodiments, the film 750 includes small apertures therein. The apertures can, for example, be smaller than the narrow, distal end portions of the helical opening 740. In such embodiments, after being trapped within the coiled section 625, the dislodged portions of the thrombus can be further ablated until they are sized to pass through the apertures of the film 750 and into the main bloodstream. Any remaining dislodged portions of the thrombus (e.g., dislodged portions of the thrombus that were too big to pass through the apertures in the film 750) can be removed from the blood vessel along with the catheter 605.

While the helical opening 740 of the coiled section 625 has been described as being wider near the proximal end of the coiled section 625 and narrower near the distal end of the coiled section 625 due to the decreasing pitch and diameter of the helical-shaped tubular member 710 distally along the coiled section 625, other configurations are possible. In some embodiments, for example, the pitch and diameter of the helical-shaped tubular member 710 increase distally along the coiled section 625 such that the width of the helical opening 740 increases in the distal direction. Alternatively, the pitch and diameter of the helical-shaped tubular member 710 can be greatest in the central portion of the coiled section 625 and decrease toward the proximal and distal ends of the coiled section 625 such that the width of the helical opening 740 is greatest near the central portion of the coiled section 625 and decreases toward the proximal and distal ends of the coiled section 625.

While the above systems has been described as using vibrational energy alone to ablate the thrombus, the coiled sections of the catheters can alternatively or additionally provide for mechanical maceration of the thrombus. During use, for example, the catheter (with the coiled section in the expanded or partially expanded state) can be moved axially back and forth within the blood vessel such that the helical-shaped tubular member(s) of the coiled section scrapes against the thrombus, which can dislodge portions of the thrombus. The systems described in FIGS. 13 and 14 above can be particularly advantageous for this type of mechanical maceration due to their ability to trap dislodged portions of the thrombus within the coiled section of the catheter.

While the proximal and distal sections of the catheters above are axially aligned with one another (i.e., lie along a straight line), in some embodiments, the proximal and distal sections of the catheter are axially offset from one another. Referring to FIG. 15, for example, a catheter 805 includes a proximal section 815, a distal section 820, and a coiled section 825 positioned between the proximal and distal sections 815, 820. The proximal and distal sections 815, 820 are axially offset from one another, causing the waveguide 135 extending through the catheter 805 to extend at an angle through the coiled section 825. As a result, when the catheter 805 is disposed within a patient's blood vessel, the portions of the waveguide 135 near the proximal and distal ends of the coiled section 825 will be positioned in close proximity to opposite surfaces of the blood vessel wall. Due to the close proximity of the portions of the waveguide 135 near the proximal and distal ends of the coiled section 825 to opposite surfaces of the blood vessel, these portions of the waveguide 135 are capable of delivering relatively intense vibrational energy to those respective surfaces of the blood vessel. As a result, it is possible to more quickly ablate a thrombus on the blood vessel wall. During use, the catheter 805 can be moved back and forth axially within the blood vessel while vibrating the waveguide 135 so that the portions of the waveguide 135 near the proximal and distal ends of the coiled section are exposed to a greater length of the vessel wall. The catheter 805 can, for example, be axially moved back and forth along the entire length of the thrombus.

The catheter 805 can be formed using a manner similar to the method described above with respect to catheter 105. However, when forming the coiled section 825 of the catheter 805, the proximal and distal sections 815, 820 are fixed in offset axial positions. FIG. 16 illustrates a device that can be used to form the catheter 805. As shown in FIG. 16, the forming device includes the mandrel 275 and sleeves 881, 883 that can be disposed around opposite end portions of the mandrel 275. The sleeves 881, 883 include axially offset bores 887, 889 that extend through end regions of the sleeves 881, 883. The bore 887 extends along a top portion of the sleeve 881 while the bore 889 extends along a bottom portion of the sleeve 883.

To form the catheter 805, the third tube 265 of the tube assembly 270 (described above with regard to FIGS. 5A and 5B) is wrapped around the mandrel 275 such that the third tube 265 is partially disposed within the helical groove 277 extending along the mandrel. The sleeves 881, 883 are then disposed over opposite end portions of the mandrel 275 and the third tube 265 to maintain the third tube 265 in the coiled configuration. The first and second tubes 255, 260 are arranged such that they extend through the axial offset bores 887, 889 in the sleeves 881, 883. As a result, the first and second tubes 255, 260 are fixed in axially offset positions relative to one another. In particular, in this configuration, the first and second tubes 255, 260 are positioned on opposite sides of the longitudinal axis of the mandrel 275 and sleeves 281, 283. With the third tube 265 held in the coiled configuration and the first and second tubes 255, 260 held in an axially offset position relative to one another, the third tube 265 is heated. The third tube 265 can, for example, use any of the various different heating techniques discussed above.

After allowing the tube assembly 270 to cool, the tube assembly 270 is removed from the mandrel 275 by pulling the sleeves 881, 883 away from one another and off of the mandrel 275 and then sliding the coiled third tube 265 off of the mandrel 275. Due to the heating process, the third tube 265 remains in a coiled configuration, and the first and second tubes 255, 260 remain axially offset from one another. The tube assembly 270 can then undergo further processing to complete the catheter 805.

The coiled sections of the catheters discussed above include multiple turns (i.e., multiple 360 degree revolutions). In some embodiments, the coiled section includes two or more (e.g., three or more, four or more, five or more, six or more, seven or more, eight or more, nine or more) turns and/or ten or fewer (e.g., nine or fewer, eight or fewer, seven or fewer, six or fewer, five or fewer, four or fewer, three or fewer, two or fewer). In certain embodiments, the coiled section includes only a single turn.

In addition, while the above catheters have been described as including only a single coiled section (e.g., the coiled section 125 of the catheter 105 and the intermediate section 525 of the catheter 505), the catheter can alternatively include multiple (e.g., two, three, four, five, or more) coiled sections along its length. Each of the coiled sections can, for example, be disposed between substantially linear segments of the catheter. Similarly, the waveguide can include multiple deformable sections (e.g., sections that form a coiled configuration when subjected to a temperature change) along its length that are disposed between substantially linear sections (e.g., sections that do not form a coiled configuration when subjected to a temperature change).

While the coiled sections of the catheters discussed above (e.g., the coiled section 125 of the catheter 105 and the intermediate section 525 of the catheter 505) have been described as surrounding the portion of the waveguide 135 that vibrates transversely during use, other arrangements are possible. The coiled section can, for example, surround portions of the waveguide that vibrate transversely during use as well as parts of the waveguide that vibrate longitudinally during use. In some embodiments, for example, the coiled section extends along a majority (e.g., 60 percent, 70 percent, 80 percent, 90 percent, 100 percent) of the length of the waveguide.

While the various sections of the above catheters have been described as being thermally bonded to one another, other attachment techniques can be used. For example, the coiled section (or the intermediate section) can be adhesively secured or mechanically fastened to the proximal and distal sections.

While the above catheters have been described as including multiple separate tubes that are bonded together, the catheters can alternatively be formed from a single tubular member. In some embodiments, for example, a straight multi-lumen tube is formed. The multi-lumen tube is then skived to create a single-lumen section along a portion of the tube with multi-lumen portions positioned at either end of the single-lumen section. The single-lumen section can then be formed into the coiled section or intermediate section of the resulting catheter while the multi-lumen sections on either end of the single-lumen section can become the proximal and distal sections of the resulting catheter. Other techniques, such as shuttle injection, can alternatively or additionally be used to form the single tubular member from which the catheter is formed. Shuttle injection can, for example, be used to form a tubular member having multiple sections with varying degrees of flexibility (e.g., multiple sections formed of different materials) along its length.

The catheter above have been described as having an opening formed in the sidewall of the proximal section of the catheter to allow the guidewire to enter or exit the guidewire lumen through the opening. Catheters having this type of configuration are often referred to as rapid-exchange catheters because the presence of the opening in the sidewall of the proximal section of the catheter (at a location spaced distally from the proximal end of the catheter) allows the user to quickly remove the catheter from the guidewire and replace it with another catheter without removing the guidewire from the patient. While the catheters above are rapid-exchange catheters, the catheters can alternatively or additionally be configured as over-the-wire catheters. In such cases, for example, the system can be configured to allow the guidewire to exit the proximal end of the catheter.

While embodiments have been described in which the cross-sectional shape of the helical-shaped tubular member(s) is/are circular, other cross-sectional shapes may also be used. For example, the cross-sectional shape of the helical-shaped tubular member(s) can be elliptical, rectangular, square, triangular, etc. In some embodiments, different portions of the catheter have different cross-sectional shapes.

While the waveguide 13 5 has been described as having multiple regions of substantially uniform cross-sectional dimension connected by tapered regions, the waveguide can alternatively be tapered along substantially its entire length. For example, the waveguide can include multiple regions along its length that taper to a smaller diameter distally. As a result, the diameter of the waveguide continuously decreases in the distal direction. The taper angle for each section can differ from the taper angle of adjacent regions. Near the distal end of the waveguide, the waveguide can taper outward to form an enlarged distal tip having a substantially uniform cross-section along its length.

While a specific example of a waveguide has been described above, other types of waveguides can alternatively or additionally be used with the above catheters. For example, while the waveguide has been described as being formed of Ti-6Al-4V titanium alloy, the waveguide can alternatively or additionally include (e.g., be formed of) one or more other materials. In general, the waveguide can be formed of any material capable of supporting ultrasonic vibrations. In some embodiments, the waveguide is formed of a material having a flexural stiffness of at least about 1×107 N/m (e.g., at least about 2.5×107 N/m, at least about 4×107 N/m) and/or at most about 10×107 N/m (e.g., at least about 8.5×107 N/m, at least about 7×107 N/m). Examples of materials from which the waveguide can be made include metals (e.g., titanium, stainless steel (e.g., 304 SS, 316 SS), etc.) and alloys (e.g., titanium alloys other than annealed Ti-6Al-4V titanium, stainless steel alloys, MP35N, 35NLT, etc.).

While embodiments have been described in which the cross-sectional shape of the waveguide 135 is circular, other cross-sectional shapes may also be used. For example, the cross-sectional shape of the waveguide 135 can be triangular, elliptical, or rectangular. In some embodiments, different portions of the waveguide 135 have different cross-sectional shapes.

While the radiopaque tip of the waveguide has been described as being formed of tantalum, the tip can alternatively or additionally include (e.g., be formed of) any of various other highly radiopaque metals or metal/polymer combinations. Examples of other radiopaque materials include platinum, gold, and alloys to platinum and/or gold. In some embodiments, the waveguide does not include a radiopaque tip.

While embodiments above describe waveguides that are transversely vibrated during use, the waveguides can alternatively or additionally be vibrated torsionally during use. The waveguide can, for example, be configured to convert longitudinal vibrations into torsional vibrations. Alternatively or additionally, the acoustic assembly can be configured to transmit torsional vibraions to the waveguide.

While the system 100 has been described as being used to ablate a thrombus within a blood vessel, the system 100 can alternatively or additionally be used for other purposes. For example, the system can be used for ablating occlusions, removing plaque, removing bone cement, treating gynecological tissue, debulking prostate, treating urolithiasis, reinforcing bone, cleaning a vascular access device, treating deep vein thrombosis (DVT), treating peripheral arterial disease, treating chronic total occlusions, phacoemulsification, and/or treating coronary thrombosis lesions.

Other embodiments are in the claims.

What is claimed is:

1. A medical system, comprising:
a catheter having a coiled section, the coiled section defining a first lumen, and the coiled section further defining a second lumen that extends axially through the coiled section; and
a waveguide disposed within the second lumen, the coiled section of the catheter being radially displaceable relative to the waveguide.

2. The medical system of claim 1, wherein the first lumen is a helical-shaped lumen.

3. The medical system of claim 2, wherein the coiled section of the catheter comprises a helical-shaped tubular member that defines the helical-shaped lumen.

4. The medical system of claim 1, wherein the catheter further comprises a proximal section disposed proximal to the coiled section and a distal section disposed distal to the coiled section, and the coiled section can be radially displaced by axially displacing at least one of the proximal and distal sections relative to the waveguide.

5. The medical system of claim 4, wherein the proximal section of the catheter defines a lumen that is substantially aligned with the first lumen of the coiled section.

6. The medical system of claim 5, wherein the distal section of the catheter defines a lumen that is substantially aligned with the first lumen of the coiled section.

7. The medical system of claim 4, wherein the proximal and distal sections of the catheter are substantially linear.

8. The medical system of claim 1, wherein the coiled section of the catheter is configured such that the coiled section assumes a first level of radial expansion when a guidewire is disposed within the first lumen and a second level of radial expansion when the guidewire is removed from the first lumen, the first level of radial expansion being less than the second level of radial expansion.

9. The medical system of claim 8, wherein the coiled section is substantially radially unexpanded when the coiled section is at the first level of radial expansion.

10. The medical system of claim 1, further comprising a guidewire disposed within the first lumen, wherein the coiled section can be radially displaced relative to the waveguide by axially displacing the guidewire relative to the coiled section.

11. The medical system of claim 10, wherein the guidewire is axially displaceable between a first position in which the guidewire is disposed in the first lumen and retains the coiled section in a radially compressed condition, and a second position in which the guidewire is removed from the first lumen and the coiled section is allowed to radially expand.

12. The medical system of claim 10, wherein the coiled section can be radially expanded relative to the waveguide by proximally displacing the guidewire relative to the coiled section.

13. The medical system of claim 1, wherein the coiled section is resiliently biased toward a radially expanded position.

14. The medical system of claim 13, wherein the coiled section is radially displaceable relative to the waveguide between the radially expanded position and a radially compressed position.

15. The medical system of claim 13, wherein the coiled section comprises one or more thermoset polymers.

16. The medical system of claim 1, further comprising an outer sheath at least partially surrounding the coiled section of the catheter, wherein the coiled section of the catheter can be radially displaced relative to the waveguide by axially displacing the outer sheath relative to the coiled section of the catheter.

17. The medical system of claim 16, wherein the outer sheath is axially displaceable between a first position in which the outer sheath at least partially surrounds the coiled section and retains the coiled section in a radially compressed condition, and a second position in which the coiled section is allowed to radially expand.

18. The medical system of claim 16, wherein the coiled section of the catheter can be radially expanded relative to the waveguide by proximally displacing the outer sheath relative to the coiled section.

19. The medical system of claim 1, wherein the coiled section of the catheter comprises a plurality of helical-shaped members.

20. The medical system of claim 1, further comprising a guidewire configured to fit within the first lumen, at least a portion of the guidewire being adapted to be formed into the shape of a coil upon heating the guidewire above a predetermined temperature.

21. The medical system of claim 20, wherein the guidewire comprises one or more shape-memory materials.

22. The medical system of claim 1, wherein a distal end portion of the waveguide is secured to the catheter.

23. The medical system of claim 22, wherein the coiled section of the catheter can be radially displaced relative to the waveguide by axially displacing the distal end portion of the waveguide.

24. The medical system of claim 1, wherein the coiled section of the catheter comprises a helical-shaped tubular member that defines a helical opening along the coiled section of the catheter.

25. The medical system of claim 24, wherein the helical-shaped tubular member is configured such that the helical opening has a first width in a first portion of the coiled section and a second width in a second portion of the coiled section, the first width being greater than the second width.

26. The medical system of claim 25, wherein the first portion of the coiled section is a proximal end region of the coiled section and the second portion of the coiled section is a distal end region of the coiled section.

27. The medical system of claim 24, further comprising a polymeric film surrounding an end portion of the coiled section.

28. A medical system, comprising:
a catheter defining a lumen; and
a guidewire configured to be disposed within the lumen of the catheter, at least a portion of the guidewire being configured to have a non-coiled configuration at a first temperature and a coiled configuration at a second temperature such that when the guidewire is at the second temperature and disposed within the lumen of the catheter, a coiled shape is imparted to the catheter.

29. The medical system of claim 28, wherein the guidewire comprises one or more shape-memory materials.

30. The medical system of claim 28, further comprising a waveguide at least partially surrounded by the catheter.

31. The medical system of claim 28, wherein the second temperature is higher than the first temperature.

32. A method, comprising:
expanding a section of a catheter within a body vessel, the expanded section of the catheter being in the form of a coil; and
vibrating a waveguide disposed within a lumen defined by the expanded section of the catheter.

33. The method of claim 32, wherein the expanded section of the catheter is disposed within an at least partially occluded region of the body vessel.

34. The method of claim 32, wherein the expanded section of the catheter comprises one or more helical-shaped tubular members.

35. The method of claim 32, wherein expanding the section of the catheter comprises proximally displacing an outer sheath relative to the section of the catheter.

36. The method of claim 35, wherein expanding the section of the catheter comprises axially displacing the outer sheath from a first position in which the outer sheath at least partially surrounds the section of the catheter and retains the section of the catheter in a radially compressed condition, toward a second position in which the outer sheath is axially spaced apart from the section of the catheter.

37. The method of claim 32, wherein expanding the section of the catheter comprises proximally displacing a guidewire relative to the section of the catheter.

38. The method of claim 37, wherein proximally displacing the guidewire comprises axially displacing the guidewire from a first position in which the guidewire is disposed within a guidewire lumen defined by the section of the catheter, toward a second position in which the guidewire is removed from the guidewire lumen defined by the section of the catheter.

39. The method of claim 32, wherein expanding the section of the catheter comprises heating or cooling a guidewire disposed within a guidewire lumen defined by the section of the catheter.

40. The method of claim 39, wherein heating the guidewire comprises passing a heated fluid through the catheter.

41. The method of claim 39, wherein heating the guidewire comprises applying electrical energy to the guidewire.

42. The method of claim 32, wherein a portion of the waveguide disposed within the expanded section of the catheter is transversely vibrated.
